(19) United States
(12) Patent Application Publication
(10) Pub. No.: US 2021/0251791 A1
(43) Pub. Date: Aug. 19, 2021

(54) Adjustable Toe Spacer

(71) Applicant: Daniel Jung, Issaquah, WA (US)

(72) Inventor: Daniel Jung, Issaquah, WA (US)

(21) Appl. No.: 16/792,225

(22) Filed: Feb. 15, 2020

(51) Int. Cl. A61F 5/01; A43B 7/26

(52) U.S. Cl. CPC A61F 5/019; A43B 7/26

(57) ABSTRACT

An adjustable toe spacer may include a flexible body that defines a first digit receptacle and a second digit receptacle. The first digit receptacle and the second digit receptacle may comprise hollow sleeves through which a first digit of a user and a second digit of a user may pass through a first digit exit and a second digit exit, respectively. The body may include a shim receptacle disposed in an area between the first digit receptacle and the second digit receptacle and configured to receive an adjustable shim. When inserted into the shim receptacle, the adjustable shim may expand the interior cavity of the shim receptacle, creating a tailored spatial extension between the first and second digit of the user.

### FIELD

This disclosure relates generally to an adjustable toe spacer.

### BACKGROUND

Foot care is crucial for overall health and well-being. Foot pain or discomfort are often signs of more serious problems. Unfortunately, foot care products on the market fail to accommodate the wide range of users that need them. For example, conventional toe spacers are not adjustable, and consumers must search for different-sized products to accommodate different toe sizes or needs. This can be costly, inconvenient, and time-consuming for users seeking relief. Users must also endure significant discomfort by wearing spacers that do not fit, which may cause further pain or injury.

### SUMMARY

The following presents a simplified summary of the disclosure to provide a basic understanding to the reader. This summary is not an extensive overview of the disclosure, nor does it identify key or critical elements of the claimed subject matter or define its scope. Its sole purpose is to present some concepts disclosed in a simplified form as a precursor to the more detailed description that is later presented.

Disclosed, among other things, is an adjustable toe spacer, which may provide an improved device and method of treatment for podiatric care. In one implementation, a flexible body may define a first digit receptacle and a second digit receptacle. The first digit receptacle and the second digit receptacle may comprise hollow sleeves through which a first digit of a user and a second digit of a user may pass, respectively. A distal end of the first digit of the user may extend through a first digit exit, and a distal end of the second digit of the user may extend through a second digit exit. The body may include a shim receptacle disposed in an area between the first digit receptacle and the second digit receptacle. The shim receptacle may define an interior cavity configured to receive an adjustable shim. When inserted into the shim receptacle, the adjustable shim may expand the interior cavity of the shim receptacle, creating a spatial extension between the first and second digit of the user. A person skilled in the art will understand that, in other implementations, the adjustable toe spacer may define a first digit receptacle configured to receive one digit or, alternatively, any number of digit receptacles to receive any number of digits.

The adjustable toe spacer may provide improved relief to a wider and more diverse set of users than conventional products by accommodating users with different toe sizes, physical characteristics, or needs. Among its many benefits, the adjustable toe spacer may be more efficient in preventing chafing or rubbing of the digits, improving blood circulation, realigning the digits, or providing another form of medical or hygienic care. A person skilled in the art will understand that the adjustable shim or any part of the adjustable toe spacer may be provided in various shapes, sizes, colors, and materials.

### BRIEF DESCRIPTION OF THE DRAWINGS

The present description may be better understood from the following detailed description read in light of the appended drawings, wherein:

FIG. 1 illustrates a perspective view of an adjustable toe spacer, according to one implementation.

FIG. 2 illustrates a perspective view of an adjustable toe spacer, according to one implementation.

FIG. 3 illustrates a front perspective view of an adjustable toe spacer, according to one implementation.

FIG. 4 illustrates a front perspective view of an adjustable toe spacer, according to one implementation.

FIG. 5 illustrates a front perspective view of an adjustable toe spacer, according to one implementation.

FIG. 6 illustrates a front perspective view of an adjustable toe spacer, according to one implementation.

FIG. 7 illustrates a front perspective view of an adjustable toe spacer, according to one implementation.

FIG. 8 illustrates a front view of an adjustable toe spacer, according to one implementation.

FIG. 9 illustrates a front perspective view of an adjustable toe spacer, according to one implementation.

FIG. 10 illustrates a flow chart showing steps in a process of an adjustable toe spacer method, according to one implementation.

### DETAILED DESCRIPTION

A more particular description of certain implementations of Adjustable Toe Spacer may be had by references to the implementations shown in the drawings that form a part of this specification, in which like numerals represent like objects.

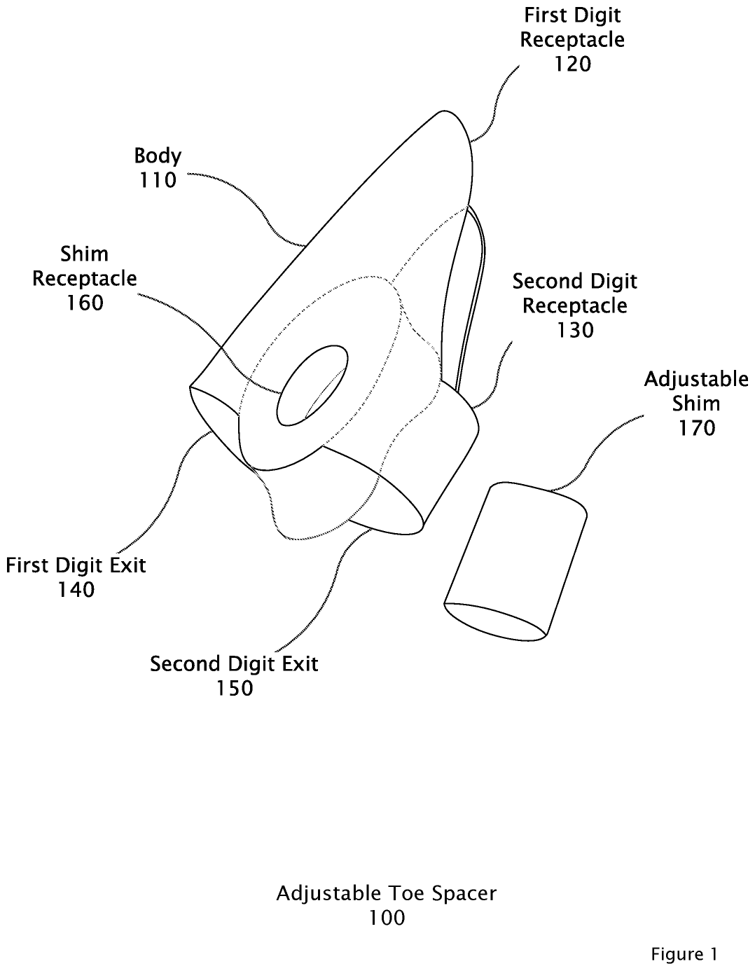

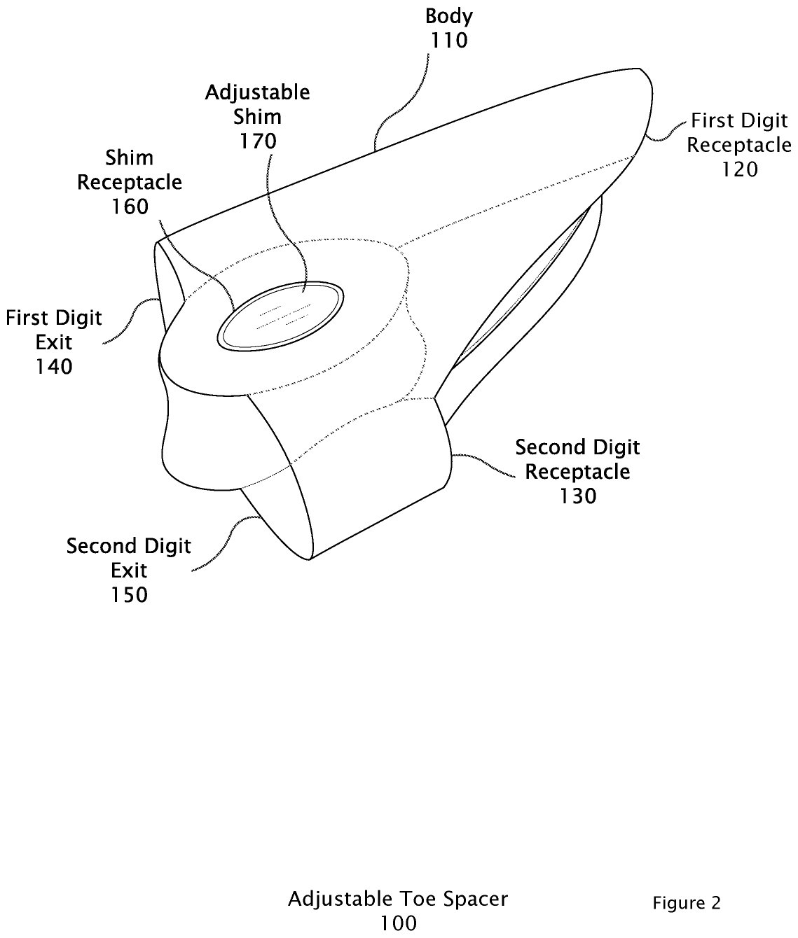

FIG. 1 and FIG. 2 illustrate perspective views of an adjustable toe spacer, according to one implementation. Adjustable Toe Spacer 100 may include a flexible Body 110 configured to fittingly receive a terminal end of an extremity, for example, a hand or a foot of a user. In this example, Body 110 may define a First Digit Receptacle 120 and a Second Digit Receptacle 130. The First Digit Receptacle 120 and the Second Digit Receptacle 130 may comprise hollow sleeves through which a first digit of a user and a second digit of a user may pass, respectively. A distal end of the first digit of the user may extend through a First Digit Exit 140, and a distal end of the second digit of the user may extend through a Second Digit Exit 150.

Body 110 may include a Shim Receptacle 160 disposed in an area between the First Digit Receptacle 120 and the Second Digit Receptacle 130. Shim Receptacle 160 may define an interior cavity configured to receive an Adjustable Shim 170 of Adjustable Toe Spacer 100. When inserted into Shim Receptacle 160, the Adjustable Shim 170 may apply lateral pressure against First Digit Receptacle 120 and Second Digit Receptacle 130, expanding the interior cavity of Shim Receptacle 160 and creating a spatial extension between the first and second digit of the user.

Adjustable Toe Spacer 100 may provide an improved device and method of treatment for podiatric care. Among many benefits, it may be more efficient in preventing chafing or rubbing of the digits, improving blood circulation, or potentially realigning the digits, for example. Adjustable Toe Spacer 110 may provide relief to a wider range of users than conventional products by accommodating users with diverse physical characteristics or treatment needs. Adjustable Toe Spacer 100 may allow a user to customize the device to the user's particular foot, toe, hand, or finger size, or to the user's particular medical or hygienic needs.

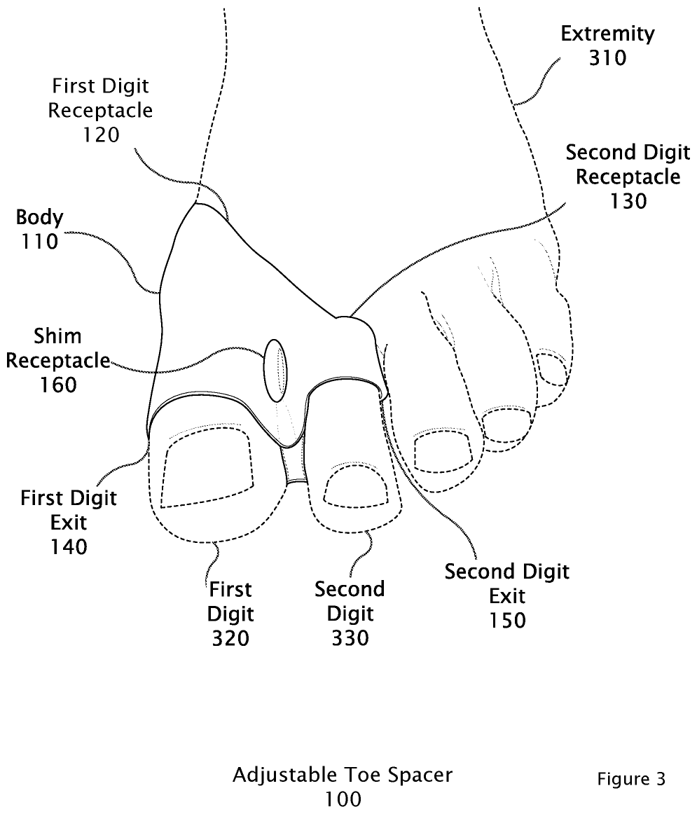

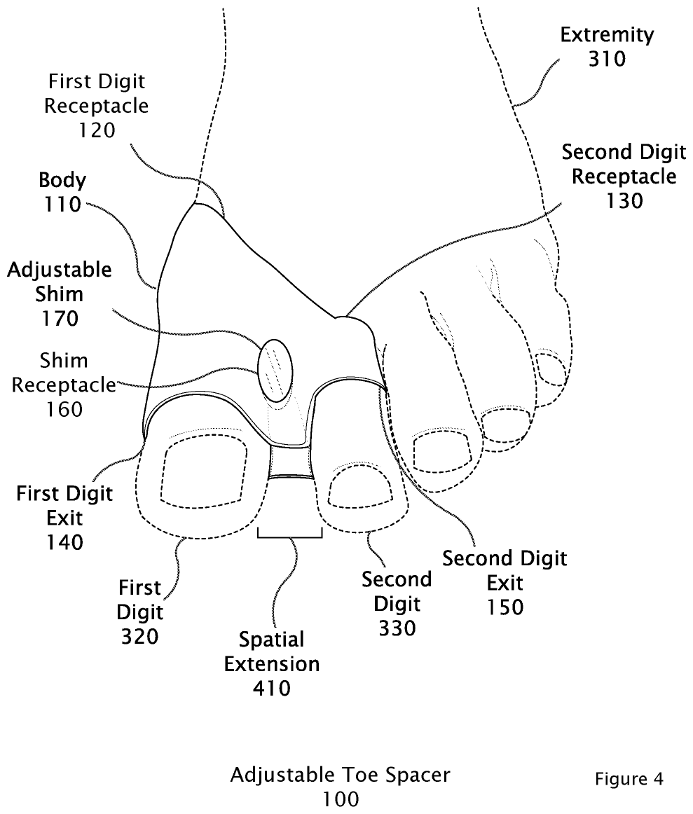

FIG. 3 and FIG. 4 illustrate front perspective views of Adjustable Toe Spacer 100, according to one implementation. Body 110 may fittingly receive a terminal end of an Extremity 310, for example, a foot, a hand, fingers, toes, or another part of a user. Adjustable Toe Spacer 100 may be configured for use on a human, on another type of animal, or on another living or non-living thing. First Digit Receptacle 120 and Second Digit Receptacle 130 may comprise hollow sleeves through which a First Digit 320 and Second Digit 330 of a user may pass, respectively. A distal end of First Digit 320 and a distal end of Second Digit 330 may extend through a First Digit Exit 140 and a Second Digit Exit 150, respectively.

Shim Receptacle 160 may be disposed between First Digit Receptacle 120 and Second Digit Receptacle 130. Shim Receptacle 160 may define an interior cavity configured to receive Adjustable Shim 170 of Adjustable Toe Spacer 100. When inserted into Shim Receptacle 160, the Adjustable Shim 170 may apply lateral pressure against First Digit Receptacle 120 and Second Digit Receptacle 130, expanding the interior cavity of Shim Receptacle 160 to create a Spatial Extension 410 between First Digit 320 and Second Digit 330. In this example, Adjustable Toe Spacer 100 may receive two digits, for example, the first toe (also known as the hallux) and the second toe (also known as the index toe). A person skilled in the art will understand that Adjustable Toe Spacer 100 may be configured to fit on any other digit, a combination of digits, or on another part of the user.

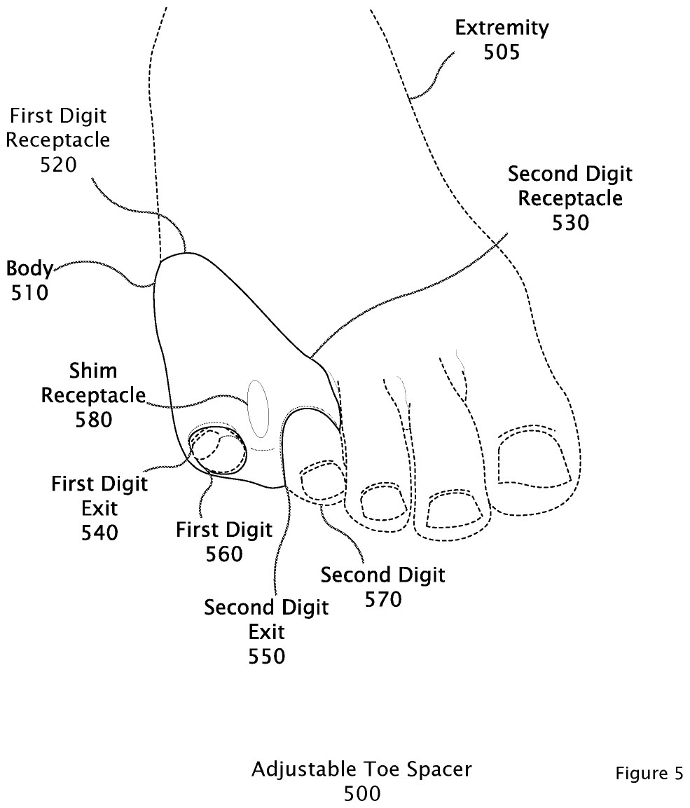

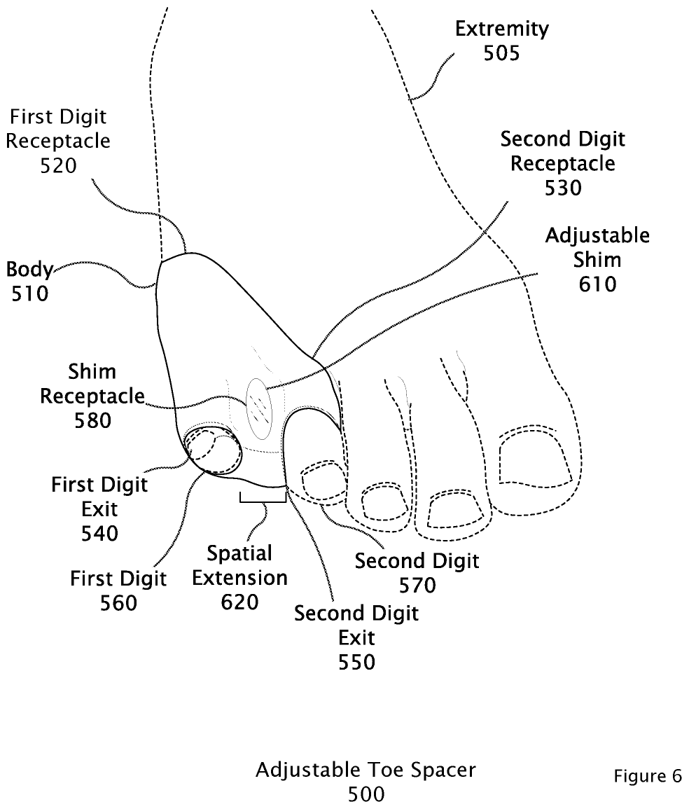

FIG. 5 and FIG. 6 illustrate front perspective views of Adjustable Toe Spacer 500, according to one implementation. In this example, First Digit 560 may comprise the fifth toe (also known as the pinky toe), and Second Digit 570 may comprise the fourth toe (also known as the ring toe). A person skilled in the art will understand that Adjustable Toe Spacer 510 may be configured to fit on any other digit, combination of digits, or on another part of the user.

Body 100 may fittingly receive a terminal end of an Extremity 505. First Digit Receptacle 520 and Second Digit Receptacle 530 may comprise hollow sleeves through which a First Digit 560 and Second Digit 570 may pass, respectively. Distal ends of First Digit 560 and Second Digit 570 may extend through First Digit Exit 540 and a Second Digit Exit 550, respectively.

Shim Receptacle 580 may be disposed between First Digit Receptacle 520 and Second Digit Receptacle 530. Shim Receptacle 580 may define an interior cavity configured to receive Adjustable Shim 610 of Adjustable Toe Spacer 500. When inserted into Shim Receptacle 580, the Adjustable Shim 610 may apply lateral pressure against First Digit Receptacle 520 and Second Digit Receptacle 530, expanding the interior cavity of Shim Receptacle 580 to create a Spatial Extension 620 between First Digit 560 and Second Digit 570.

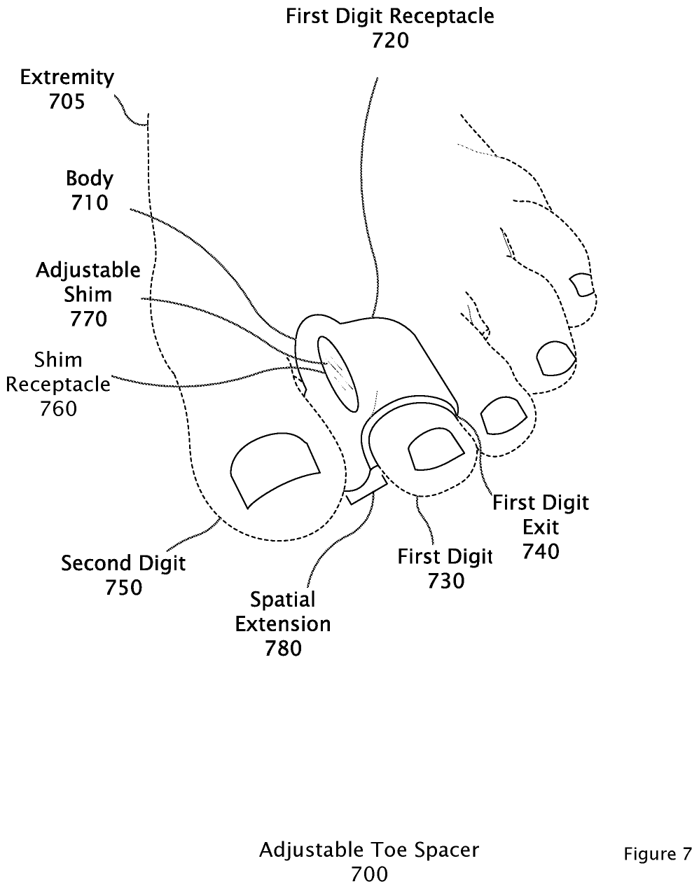

FIG. 7 illustrates a front perspective view of an Adjustable Toe Spacer 700, according to one implementation. In this example, Body 710 may receive First Digit 730 of Extremity 705. First Digit 730 may pass through First Digit Receptacle 720 and extend through First Digit Exit 740. Shim Receptacle 760 may be disposed in an area between First Digit Receptacle 720 and Second Digit 750. When inserted into Shim Receptacle 760, Adjustable Shim 770 may create Spatial Extension 780 between First Digit 730 and Second Digit 750 of the user. In this example, First Digit 730 may comprise second toe (also known as the index toe). Adjustable Toe Spacer 700 may create Spatial Extension 780 between the second toe and the first toe (also known as the hallux), or between any other digits or parts of the user.

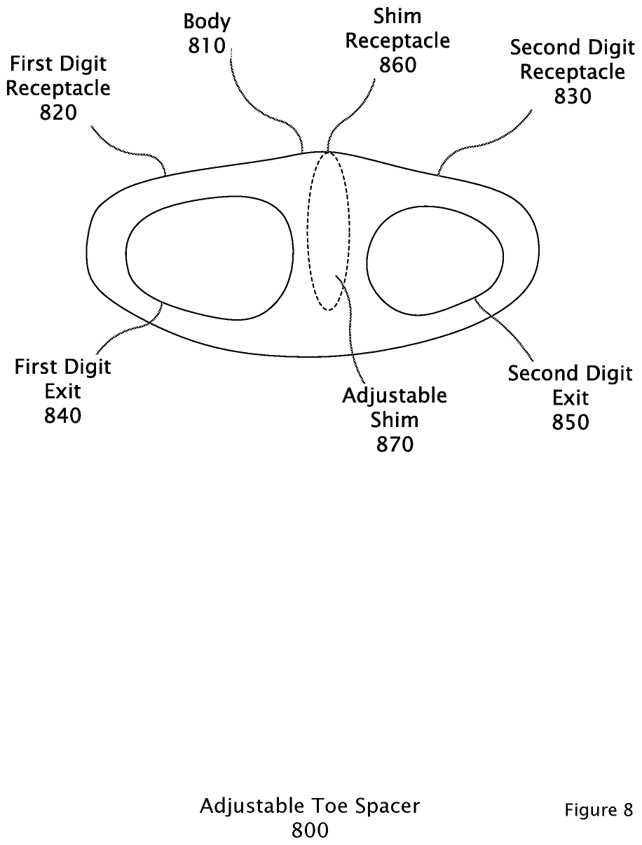

FIG. 8 illustrates a front view of Adjustable Toe Spacer 800, according to one implementation. Body 810 may define First Digit Receptacle 820, Second Digit Receptacle 830, First Digit Exit 840, and Second Digit Exit 850. Shim Receptacle 860 may comprise an interior cavity to receive Adjustable Shim 870. A person skilled in the art will understand that Adjustable Shim 870 or any part of Adjustable Toe Spacer 800 may be provided in various shapes, sizes, colors, and materials. For example, Adjustable Toe Spacer 800 and Adjustable Shim 870 may be made of TPE (Thermoplastic Elastomer) gel, silicone, foam, plastic, nylon, cotton, or a soft or elastic material. In one implementation, a user may trim down or otherwise modify Adjustable Shim 870 to customize a thickness. In another implementation, Adjustable Shim 870 may include a spring, or it may be made of an expandable or contractable material, for example.

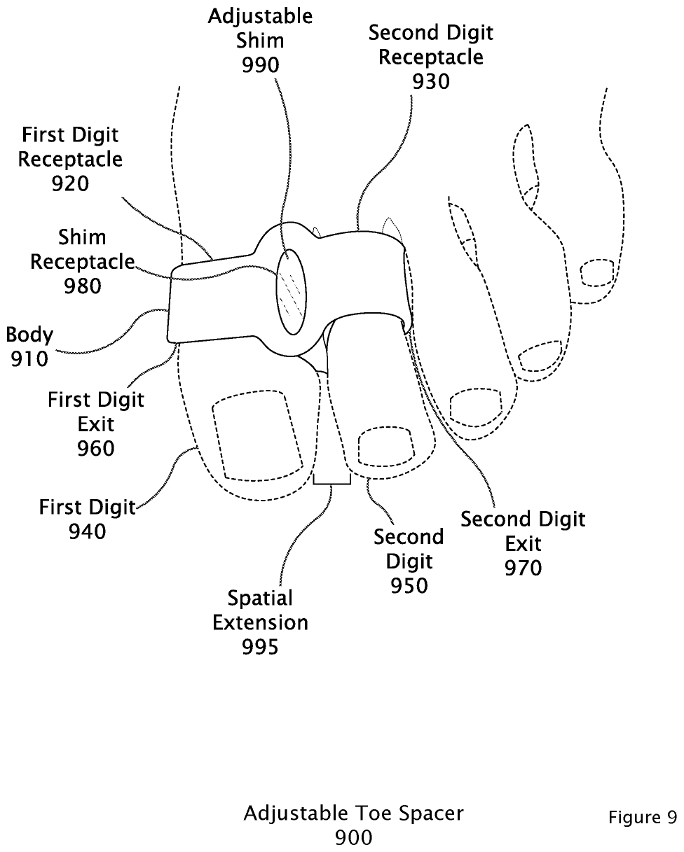

FIG. 9 illustrates a front perspective view of Adjustable Toe Spacer 900, according to one implementation. Body 910 may define a First Digit Receptacle 920 and a Second Digit Receptacle 930, which may comprise one or more rings configured to receive a First Digit 940 and a Second Digit 950, respectively. A distal end of First Digit 940 and a distal end of Second Digit 950 may extend through a First Digit Exit 960 and a Second Digit Exit 970, respectively. Shim Receptacle 980 may be disposed between First Digit Receptacle 920 and Second Digit Receptacle 930. Shim Receptacle 980 may define an interior cavity configured to receive Adjustable Shim 990 of Adjustable Toe Spacer 900. When inserted into Shim Receptacle 980, the Adjustable Shim 990 may apply lateral pressure against First Digit Receptacle 920 and Second Digit Receptacle 930, expanding the interior cavity of Shim Receptacle 980 to create a Spatial Extension 995 between First Digit 940 and Second Digit 950. In this example, Adjustable Toe Spacer 100 may receive two digits, for example, the first toe (also known as the hallux) and the second toe (also known as the index toe). A person skilled in the art will understand that Adjustable Toe Spacer 900 may be configured to fit on any other digit, combination of digits, or on another part of the user.

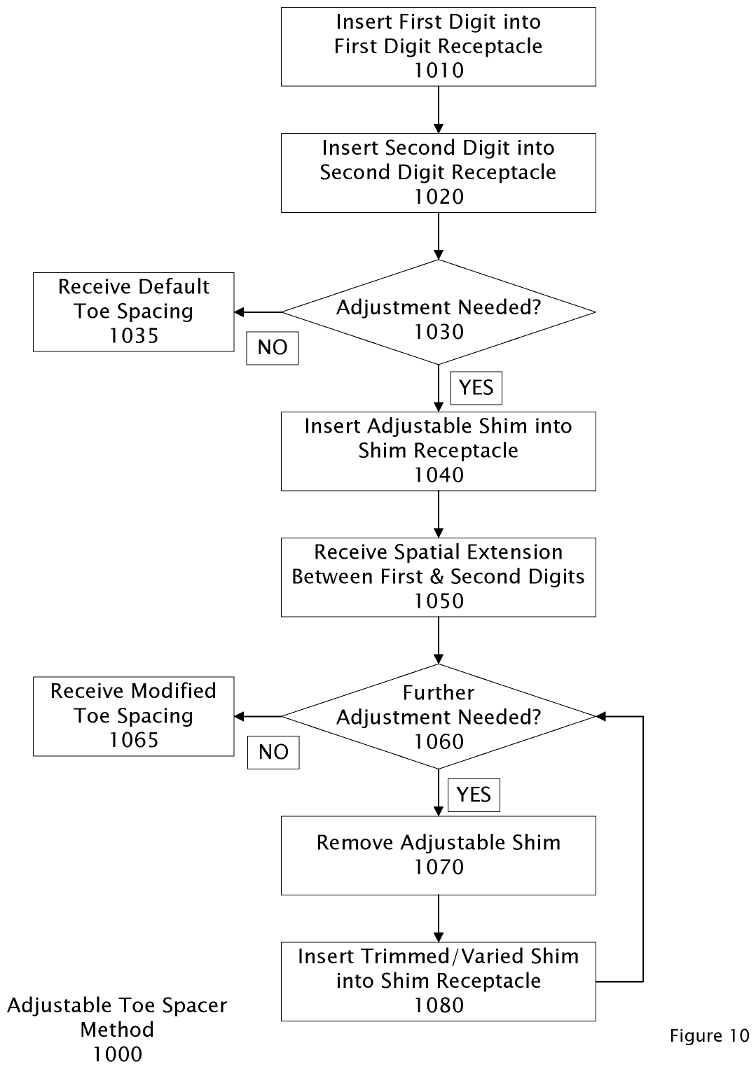

FIG. 10 illustrates a flow chart showing steps in a process of an Adjustable Toe Spacer Method 1000, according to one implementation. At Insert First Digit into First Digit Receptacle 1010, the adjustable toe spacer may receive a first digit of a user in a first digit receptacle. At Insert Second Digit into Second Digit Receptacle 1020, the adjustable toe spacer may receive a second digit of the user in a second digit receptacle.

At Adjustment Needed? 1030, if the user needs a more personalized fit to accommodate a physical characteristic or need of the user, for example, the user may proceed to YES, to Insert Adjustable Shim into Shim Receptacle 1040. If NO, the user does not need an adjustment, then the user may proceed to Receive Default Toe Spacing 1035.

At Insert Adjustable Shim into Shim Receptacle 1040, the user may insert an adjustable shim into the shim receptacle, causing the adjustable shim to expand an interior cavity of the shim receptacle to apply lateral pressure against the first digit receptacle and the second digit receptacle. At Receive Spatial Extension Between First and Second Digits 1050, the lateral pressure may expand the interior cavity of the shim receptacle, creating a spatial extension between the first and second digit of the user.

At Further Adjustment Needed? 1060, the user may determine whether he or she needs a more tailored fit. If YES, then the user may proceed to Remove Adjustable Shim 1070. If NO, the user does not need a further adjustment, then the user may proceed to Receive Modified Toe Spacing 1065.

If the user chose YES, then the user may remove the adjustable shim from the shim receptacle by any means, for example, by using a finger to remove it, by pressing on the removal shim or popping it out, or by using a tool to remove the shim, among many possible modes of removal. The user may select a shim having a different size, shape, material, or other characteristic, or the user may select the same shim after trimming it down to achieve a varied thickness, or otherwise modifying the shim. For example, the adjustable shim may be trimmed depending on an amount of separation that the user needs. The user may proceed to Insert Trimmed/Varied Shim into Shim Receptacle 1080 before re-assessing whether a more tailored fit is needed at Further Adjustment Needed? 1060. If NO, then the user may proceed to Receive Modified Toe Spacing 1065. If YES, then the user may continue to trim, replace, or otherwise modify the shim in steps 1070 and 1080 until a desired fit is achieved.

Unless defined otherwise, the technical and scientific terms used herein have the same meaning as commonly understood by one of ordinary skill in the relevant art. Although any methods and materials similar or equivalent to those described herein may also be used in the practice or testing of the present claims, a necessarily limited number of the exemplary methods and materials are described herein.

The terms “include” and “comprise” as well as derivatives and variations thereof, in all of their syntactic contexts, are to be construed without limitation in an open, inclusive sense, (e.g., “including, but not limited to”). The term “or,” is inclusive, meaning and/or.

While the detailed description above has been expressed in terms of specific examples, those skilled in the art will appreciate that many other configurations could be used. Accordingly, it will be appreciated that various equivalent modifications of the above-described implementations may be made without departing from the spirit and scope of the invention.

Additionally, the illustrated operations in the description show certain events occurring in a certain order. In alternative implementations, certain operations may be performed in a different order, modified or removed. Moreover, steps may be added to the above-described logic and still conform to the described implementations. Further, operations described herein may occur sequentially, or certain operations may be processed in parallel.

1. An adjustable toe spacer, comprising:
a flexible body, the flexible body operable to fittingly receive a terminal end of an extremity, whereby the flexible body defines a first digit receptacle and a second digit receptacle;
a first digit receptacle, the first digit receptacle comprising a hollow sleeve operable to receive a first digit of a user;
a second digit receptacle, the second digit receptacle comprising a hollow sleeve operable to receive a second digit of the user;
a first digit exit, the first digit exit disposed at a distal end of the first digit receptacle;
a second digit exit disposed at a distal end of the second digit receptacle;
a shim receptacle, the shim receptacle disposed in an area between the first digit receptacle and the second digit receptacle, whereby the shim receptacle defines an interior cavity operable to receive an adjustable shim; and
an adjustable shim, the adjustable shim operable to be inserted into the interior cavity of the shim receptacle to apply lateral pressure against the first digit receptacle and the second digit receptacle, expanding the interior cavity of the shim receptacle and creating a spatial extension between the first digit and the second digit of the user.

2. The adjustable toe spacer of claim 1, comprising a material selected from the list consisting of thermoplastic elastomer gel, silicone, foam, plastic, nylon, and cotton.

3. The adjustable toe spacer of claim 1, further comprising a material selected from the list consisting of a spring, an expandable material, and a contractable material.

4. The adjustable toe spacer of claim 1, wherein the adjustable shim is operable to be trimmed or otherwise modified in size to provide a more tailored fit to the user.

5. An adjustable toe spacer, comprising:
a flexible body, the flexible body operable to fittingly receive a terminal end of an extremity, whereby the flexible body defines a first digit receptacle, the first digit receptacle comprising a hollow sleeve operable to receive a first digit of a user;
a first digit exit, the first digit exit disposed at a distal end of the first digit receptacle;
a shim receptacle, the shim receptacle disposed in an area between the first digit receptacle and a second digit of the user; and
an adjustable shim, the adjustable shim operable to be inserted into the interior cavity of the shim receptacle to apply lateral pressure against the first digit receptacle and the second digit of the user, expanding the interior cavity of the shim receptacle and creating a spatial extension between the first digit and the second digit of the user.

6. The adjustable toe spacer of claim 5, comprising a material selected from the list consisting of thermoplastic elastomer gel, silicone, foam, plastic, nylon, and cotton.

7. The adjustable toe spacer of claim 5, further comprising a material from the list which includes a spring, an expandable material, and a contractable material.

8. The adjustable toe spacer of claim 5, wherein the adjustable shim is operable to be trimmed or otherwise modified in size to provide a more tailored fit to the user.
